(19) United States
(12) Patent Application Publication
(10) Pub. No.: US 2025/0154541 A1
(43) Pub. Date: May 15, 2025

(54) Bacillus Amyloliquefaciens Strain MHDS-1 And Use Thereof In Processing Of Taxus Media

(71) Applicant: Henan University of Chinese Medicine, Zhengzhou (CN)

(72) Inventors: Zhenling ZHANG, Zhengzhou (CN); Xing GUO, Zhengzhou (CN); Hanting JIA, Zhengzhou (CN); Yishuo WANG, Zhengzhou (CN); Qingya LI, Zhengzhou (CN)

(21) Appl. No.: 18/939,614

(22) Filed: Nov. 7, 2024

(30) Foreign Application Priority Data
Nov. 10, 2023 (CN) 202311490124.8

(51) Int. Cl. C12P 17/02; C12N 1/20; C12R 1/07

(52) U.S. Cl. CPC C12P 17/02; C12N 1/20; C12R 2001/07

(57) ABSTRACT

A Bacillus amyloliquefaciens strain MHDS-1 and use thereof in processing of Taxus media are provided, belonging to the technical field of microorganisms. The Bacillus amyloliquefaciens strain MHDS-1 herein is deposited in the China Center for Type Culture Collection (CCTCC) with a deposit number of CCTCC NO: M20231262. The strain is used to process the Taxus media, and can effectively increase a paclitaxel content of the Taxus media, which is not less than 20% higher than that of unprocessed Taxus media.

### CROSS REFERENCE TO RELATED APPLICATION

This patent application claims the benefit and priority of Chinese Patent Application No. 202311490124.8 filed with the China National Intellectual Property Administration on Nov. 10, 2023, the disclosure of which is incorporated by reference herein in its entirety as part of the present application.

### REFERENCE TO SEQUENCE LISTING

A computer readable XML file entitled “GWP20240705245”, that was created on Sep. 26, 2024, with a file size of about 3,585 bytes, contains the sequence listing for this application, has been filed with this application, and is hereby incorporated by reference in its entirety.

### TECHNICAL FIELD

The present disclosure belongs to the technical field of microorganisms, and in particular relates to a Bacillus amyloliquefaciens strain MHDS-1 and use thereof in processing of Taxus media.

### BACKGROUND

Paclitaxel has anti-tumor effects and is mainly extracted directly from plants, and can also be obtained through microbial transformation. Taxus media is a shrub of the genus Taxus, has rich endophytes, and can produce a variety of anti-tumor active ingredients such as paclitaxel, 10-deacetylbaccatin III, baccatin III, and cephalomannine. The Taxus media is a national first-class protected plant in China with scarce wild resources and a long growth period for cultivated products. Currently, cultivated Taxus media is mostly used to directly extract the paclitaxel, or directly used in clinical practice after cleaning and cutting. According to the syndrome differentiation, an appropriate symptomatic treatment prescription is selected and decocted with Taxus media decoction pieces for the treatment of tumors.

Traditional Chinese medicine fermentation refers to use of microorganisms and enzymes to allow catalysis and decomposition to achieve enhancing efficacy, reducing toxicity, or producing new efficacy. Studies have shown that the content of effective ingredients in the traditional Chinese medicine can be increased after fermentation-based processing. In the prior art (LIU Qiujin, YIN Junyi, ZHANG Jun, et al. Changes of effective ingredients in solid-state fermentation of Radix Astragali and Radix Isatidis [J]. Modern Animal Husbandry Science & Technology, 2022 (04): 9-11.), solid-state fermentation of Radix Astragali and Radix Isatidis has been conducted on Bacillus amyloliquefaciens, thereby increasing contents of total polysaccharides and total saponins in the Radix Astragali and Radix Isatidis. However, for different traditional Chinese medicine, unreasonable selection of strains may affect the fermentation effect, or even make fermentation impossible. At present, the only processing method for the Taxus media decoction pieces is cleaning and cutting, and there is no other processing method for the Taxus media. Therefore, it is extremely important for saving Taxus media resources by developing novel decoction pieces through establishing a fermentation-based processing method of the Taxus media.

So far, there is no strain for solid fermentation of Taxus media. As a result, it is of great significance for the development of novel traditional Chinese medicine decoction pieces by developing a new Bacillus amyloliquefaciens strain that can effectively increase the content of anti-tumor active ingredients in Taxus media.

### SUMMARY

To overcome the shortcomings of the prior art, the present disclosure aims to provide a Bacillus amyloliquefaciens strain MHDS-1 and use thereof in processing of Taxus media. After processing the Taxus media, a paclitaxel content therein can be effectively increased.

The present disclosure provides a Bacillus amyloliquefaciens strain MHDS-1, where the Bacillus amyloliquefaciens strain MHDS-1 is deposited in the China Center for Type Culture Collection (CCTCC) with a deposit number of CCTCC NO: M20231262.

The present disclosure further provides use of the Bacillus amyloliquefaciens strain MHDS-1 and/or a bacterial suspension comprising the Bacillus amyloliquefaciens strain MHDS-1 in processing of Taxus media.

The present disclosure further provides use of the Bacillus amyloliquefaciens strain MHDS-1 and/or a bacterial suspension comprising the Bacillus amyloliquefaciens strain MHDS-1 in increasing a paclitaxel content of Taxus media.

The present disclosure further provides a processing method of Taxus media, including subjecting the Taxus media to fermentation with the Bacillus amyloliquefaciens strain MHDS-1 according to the above technical solution.

Preferably, the processing method includes:

mixing an activated Bacillus amyloliquefaciens strain MHDS-1, the Taxus media, wheat bran, flour, and water to allow fermentation culture.

Preferably, the method for activating Bacillus amyloliquefaciens includes: inoculating the Bacillus amyloliquefaciens strain MHDS-1 on an activation medium, and culturing at 32° C. to 35° C. for 32 h to 38 h to prepare the activated Bacillus amyloliquefaciens strain MHDS-1.

Preferably, a total mass of the Taxus media, the wheat bran, the flour, and the water and a viable count of the Bacillus amyloliquefaciens strain MHDS-1 are at a ratio of 100 g:0.2×10⁸ CFU to 100 g:0.9×10⁸ CFU;

the Taxus media, the wheat bran, the flour, and the water are at a dosage ratio of 80-120 g:8-20 g:4-10 g:70-110 mL; and

the fermentation culture is conducted at 29° C. to 33° C. under a humidity of 65% to 75% for 60 h to 84 h.

The present disclosure further provides a traditional Chinese medicine decoction piece of Taxus media prepared by drying a fermentation product processed by the processing method.

Preferably, the drying is conducted at 55° C. to 65° C.

The present disclosure further provides use of the traditional Chinese medicine decoction piece of the Taxus media in preparation of an anti-tumor product.

### Beneficial Effects

The present disclosure provides a Bacillus amyloliquefaciens strain MHDS-1, where the strain is deposited in the CCTCC with a deposit number of CCTCC NO: M20231262. The strain is used to process the Taxus media, and can effectively increase a paclitaxel content of the Taxus media, which is not less than 20% higher than that of an unprocessed sample.

In the present disclosure, the processing method of the Taxus media has high repeatability, a quality of the obtained fermentation product is stable, and the preparation process of Taxus media slices is simplified. The processing method can effectively increase a content of the anti-tumor effective ingredients of Taxus media, and alleviate the shortage of Taxus media resources.

### Information of Biological Deposit

The Bacillus amyloliquefaciens strain MHDS-1 has been deposited in the China Center for Type Culture Collection (CCTCC) in Wuhan University, China on Jul. 10, 2023, with a deposit number of CCTCC NO: M20231262.

### BRIEF DESCRIPTION OF THE DRAWINGS

To describe the technical solutions in the examples of the present disclosure or in the prior art more clearly, the drawings required for the examples will be briefly described below.

FIG. 1 shows a total ion chromatogram (TIC) of the baccatin III standard;

FIG. 2 shows a TIC of the MHDS-1 fermentation broth extract;

FIG. 3 shows a first-order mass spectrometry (MS) diagram of the baccatin III standard;

FIG. 4 shows a first-order mass spectrometry (MS) diagram of the MHDS-1 fermentation broth extract;

FIG. 5 shows a second-order mass spectrometry (MS/MS) diagram of the baccatin III standard;

FIG. 6 shows a second-order mass spectrometry (MS/MS) diagram of the MHDS-1 fermentation broth extract;

FIG. 7 shows a plate image of the strain MHDS-1;

FIG. 8 shows a 1000× micrograph of the strain MHDS-1;

FIG. 9 shows a diagram of the whole genome of the strain MHDS-1 calculated using OAT software.

### DETAILED DESCRIPTION OF THE EMBODIMENTS

The present disclosure provides a Bacillus amyloliquefaciens strain MHDS-1, where the strain is deposited in the CCTCC with a deposit number of CCTCC NO: M20231262.

In the present disclosure, the Bacillus amyloliquefaciens strain MHDS-1 is isolated from the bark of three-year-old Taxus media, and can increase a paclitaxel content in the Taxus media. The results of examples show that the strain is used to process the Taxus media, and can effectively increase a paclitaxel content of the Taxus media, which is not less than 20% higher than that of a non-fermented and unprocessed sample.

Based on the effect of the Bacillus amyloliquefaciens strain MHDS-1, use of the Bacillus amyloliquefaciens strain MHDS-1 and/or a bacterial suspension comprising the Bacillus amyloliquefaciens strain MHDS-1 in processing of Taxus media and in increasing a paclitaxel content of the Taxus media also falls within the protection scope of the present disclosure.

The present disclosure further provides a processing method of Taxus media, including subjecting the Taxus media to fermentation with the Bacillus amyloliquefaciens strain MHDS-1.

In the present disclosure, the Bacillus amyloliquefaciens strain MHDS-1 is preferably inoculated on an activation medium to allow activation culture to obtain an activated Bacillus amyloliquefaciens strain MHDS-1. The activation culture is conducted at preferably 32° C. to 35° C., more preferably 33° C. for preferably 32 h to 38 h, more preferably 36 h. In the present disclosure, the activation medium is preferably an LB solid medium, including 10 g/L of peptone, 5 g/L of yeast extract, 10 g/L of sodium chloride, 15 g/L of agar, and water as a balance. Each component is dissolved by heating and then sterilized by high-pressure steam at 121° C. for 30 min.

In the present disclosure, after the activated Bacillus amyloliquefaciens MHDS-1 is obtained, the activated Bacillus amyloliquefaciens strain MHDS-1, the Taxus media, wheat bran, flour, and water are mixed to allow fermentation culture.

In the present disclosure, a total mass of the Taxus media, the wheat bran, the flour, and the water and a viable count of the Bacillus amyloliquefaciens strain MHDS-1 are at a ratio of preferably 100 g:0.2×10⁸ CFU to 100 g:0.9×10⁸ CFU, more preferably 100 g: 0.9×10⁸ CFU. The Taxus media, the wheat bran, the flour, and the water are at a dosage ratio of preferably 80-120 g:8-20 g:4-10 g:70-110 mL, more preferably 80 g:10 g:6 g:70 mL or 100 g:20 g:10 g:110 mL, and even more preferably 100 g:20 g:10 g:110 mL. Preferably, a fine powder of the Taxus media is used as a raw material, and the fine powder has a particle size of preferably 80 mesh to 100 mesh.

In the present disclosure, a process of mixing the activated Bacillus amyloliquefaciens strain MHDS-1, the Taxus media, the wheat bran, the flour, and the water preferably includes: mixing the Taxus media and the wheat bran to obtain a medical powder; adding the water into the flour, boiling and allowing cool to a room temperature to obtain a paste; blending the paste and the medical powder evenly and then mixing a resulting mixture with the activated Bacillus amyloliquefaciens strain MHDS-1.

In the present disclosure, before the activated Bacillus amyloliquefaciens strain MHDS-1 is mixed with the paste and the medical powder, colonies are preferably scraped into sterile water with an inoculation loop, and a resulting mixture is shaken to obtain a bacterial suspension comprising the Bacillus amyloliquefaciens strain MHDS-1. The bacterial suspension is then mixed with other ingredients; the Bacillus amyloliquefaciens strain MHDS-1 in the bacterial suspension has a concentration of preferably 1.5×10⁸ CFU/mL. A preparation process of the sterile water preferably includes: sterilizing distilled water with high-pressure steam at 121° C. for 30 min.

In the present disclosure, the fermentation culture is conducted at preferably 29° C. to 33° C., more preferably 33° C. under a humidity of preferably 65% to 75%, more preferably 75% for preferably 60 h to 84 h, more preferably 60 h. The fermentation of Taxus media with the Bacillus amyloliquefaciens strain MHDS-1 shows stable quality and high repeatability, and can effectively increase the paclitaxel content in Taxus media.

The present disclosure further provides a traditional Chinese medicine decoction piece of Taxus media prepared by drying a fermentation product processed by the processing method.

In the present disclosure, the drying is conducted at preferably 55° C. to 65° C., more preferably 65° C. There is no specific requirement for a drying time, and the drying time is sufficient until a broken medicine block has a loose cross-section and a water content of less than 10%. The drying is preferably oven drying.

The present disclosure further provides use of the traditional Chinese medicine decoction piece of the Taxus media in preparation of an anti-tumor product.

In order to further illustrate the present disclosure, the Bacillus amyloliquefaciens strain MHDS-1 and the use thereof in processing of Taxus media provided by the present disclosure are described in detail below with reference to the drawings and examples, but the drawings and the examples should not be construed as limiting the protection scope of the present disclosure.

### Example 1

### Isolation, Identification, and Deposit of the Strain

### 1. Isolation

A strain was isolated, purified, and cultured from the bark of three-year-old Taxus media obtained from the cultivation base of Pingdingshan Zhiyuan Biotechnology Co., Ltd., and was numbered M-SHDS-22.

### 2. Identification and Deposit

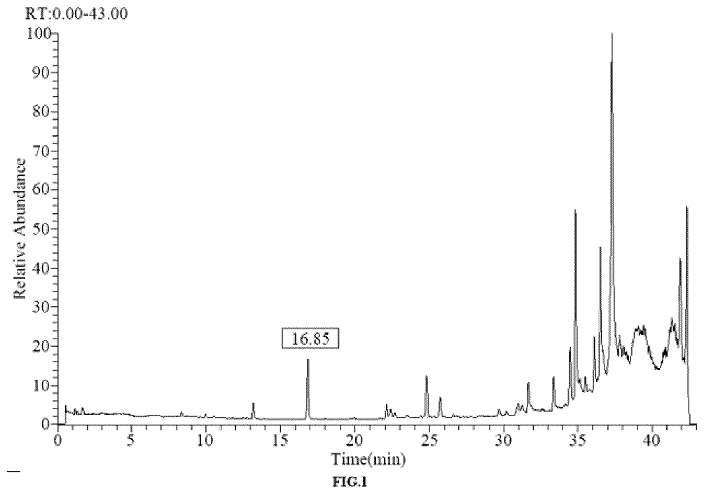

Ultra Performance Liquid Chromatography-Mass Spectrometry (UPLC-MS) identification: 37 g of a PDA powder was added into 1 L of deionized water and boiled until clear and transparent, and then sterilized in an autoclave at 121° C. for 30 min to obtain an activation medium. The strain M-SHDS-22 was inoculated into the activation medium and activated in a biochemical incubator, and a certain amount of bacterial blocks were taken out using a sterile puncher and inoculated into a broth to allow full shaking culture in the dark. An obtained culture product was filtered under reduced pressure using a Buchner funnel, and an obtained bacterial solution was separated from an obtained solid mycelium. The solid mycelium was ultrasonically extracted using ethyl acetate, and a resulting organic phase was collected; the bacterial solution was extracted by shaking with an equal volume of ethyl acetate, and a resulting organic phase was collected. The organic phases of mycelium and bacterial solution were combined, filtered through double-layer filter paper, evaporated to dryness under reduced pressure. A resulting product was dissolved in chromatographic methanol, and passed through an organic filter membrane. An obtained filtrate was divided into centrifuge tubes, vacuum concentrated and evaporated to dryness. A resulting product was re-dissolved in mass spectrometry grade methanol, centrifuged, and a supernatant was taken and filtered through an organic filter membrane to obtain a fermentation broth. Using baccatin III as a control, the fermentation broth of strain M-SHDS-22 and the baccatin III were detected using Ultimate 3000-Orbitrap Exploris 240 LC/MS, and the results were shown in FIG. 1 to FIG. 6.

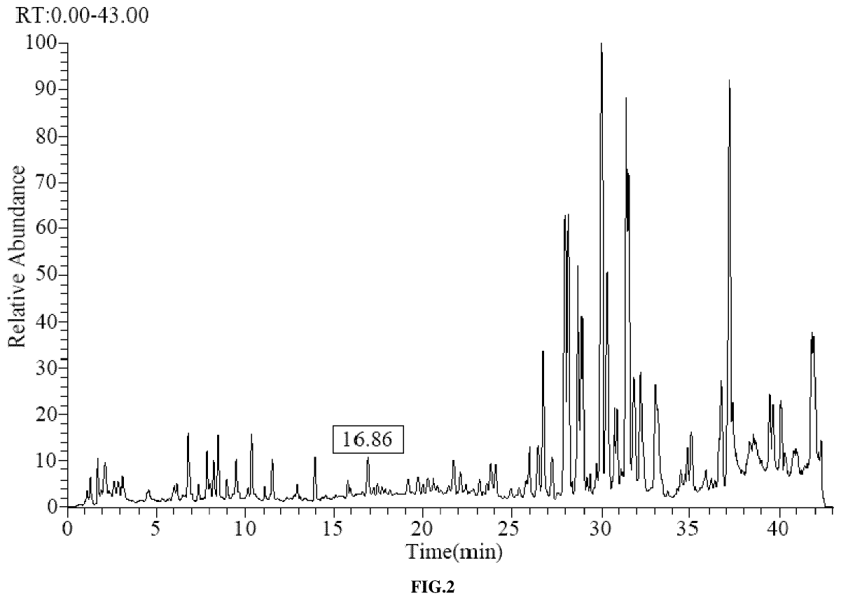

FIG. 1 showed the TIC of the baccatin III standard, where a frame line showed 16.85 as a retention time of the baccatin III standard. FIG. 2 showed the TIC of the MHDS-1 fermentation broth extract, where a frame line showed 16.86 as a retention time, which was basically consistent with that of the baccatin III standard in FIG. 1.

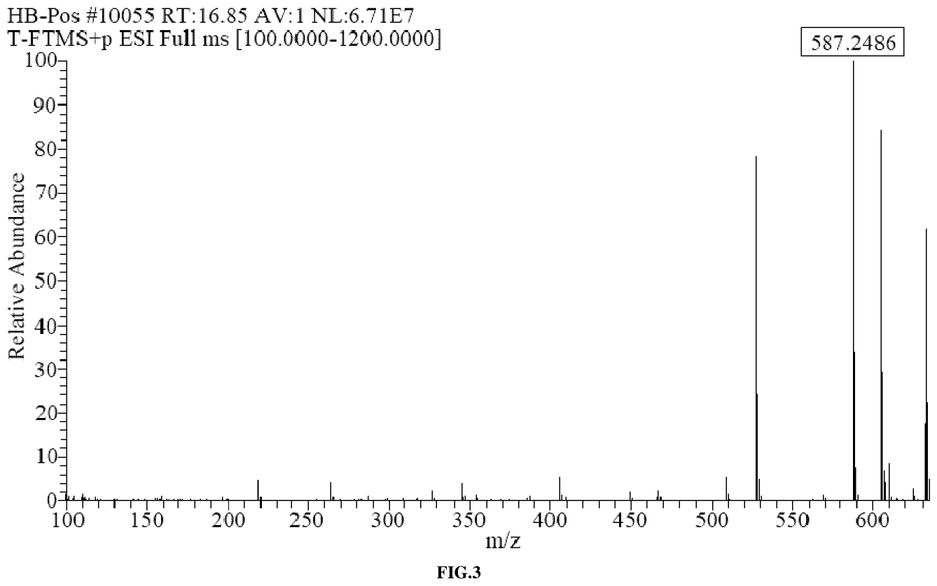

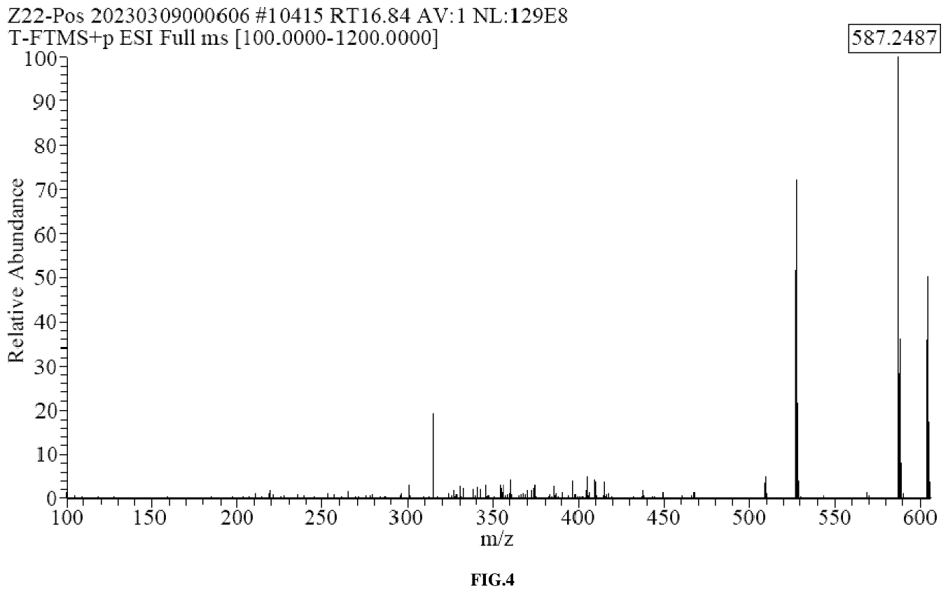

FIG. 3 showed the primary mass spectrometry (MS) diagram of the baccatin III standard, where a frame line showed a molecular ion peak m/z of the baccatin III standard, which was 587.2486 ([M+H]⁺). FIG. 4 showed the first-order mass spectrometry (MS) diagram of the MHDS-1 fermentation broth extract, where a frame line showed a molecular ion peak m/z of the MHDS-1 fermentation broth extract, which was 587.2487 ([M+H]⁺) and was basically consistent with that of the baccatin III standard in FIG. 3.

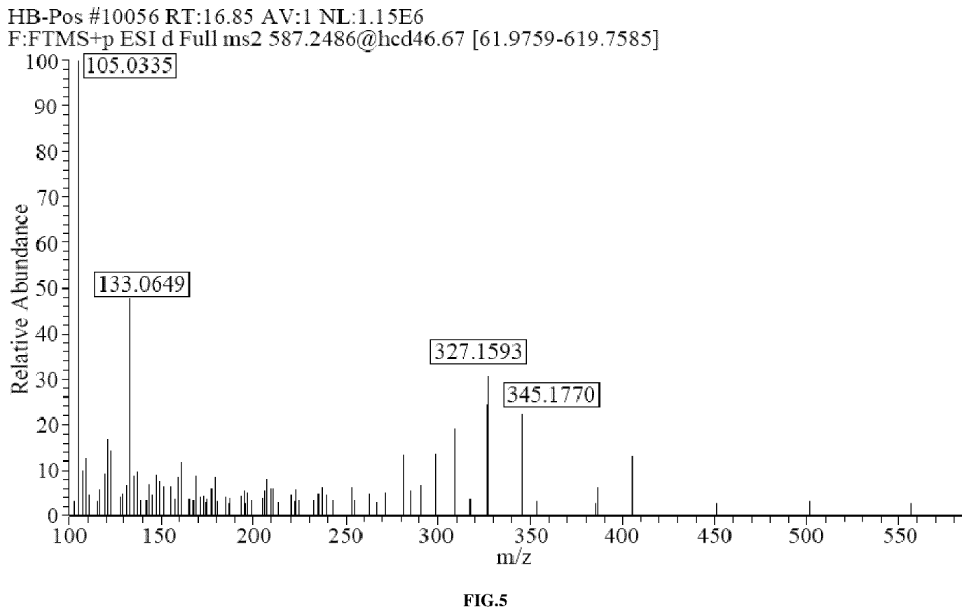

FIG. 5 showed the second-order mass spectrometry (MS/MS) diagram of the baccatin III standard, where frame lines showed 105.0335, 133.0649, 327.1593, and 345.1700 as fragment ions generated by the baccatin III standard. FIG. 6 showed the second-order mass spectrometry (MS/MS) of the MHDS-1 fermentation broth extract, where frame lines showed 105.0334, 133.0648, 327.1589, and 345.1697 as fragment ions generated by the MHDS-1 fermentation broth extract, which were also at the same level as those in FIG. 5.

In summary, the fermentation broth extract of the strain M-SHDS-22 had a retention time, parent ions, and fragment ions of second-order that were consistent with those of the baccatin III reference substance, such that the strain was identified as producing baccatin III.

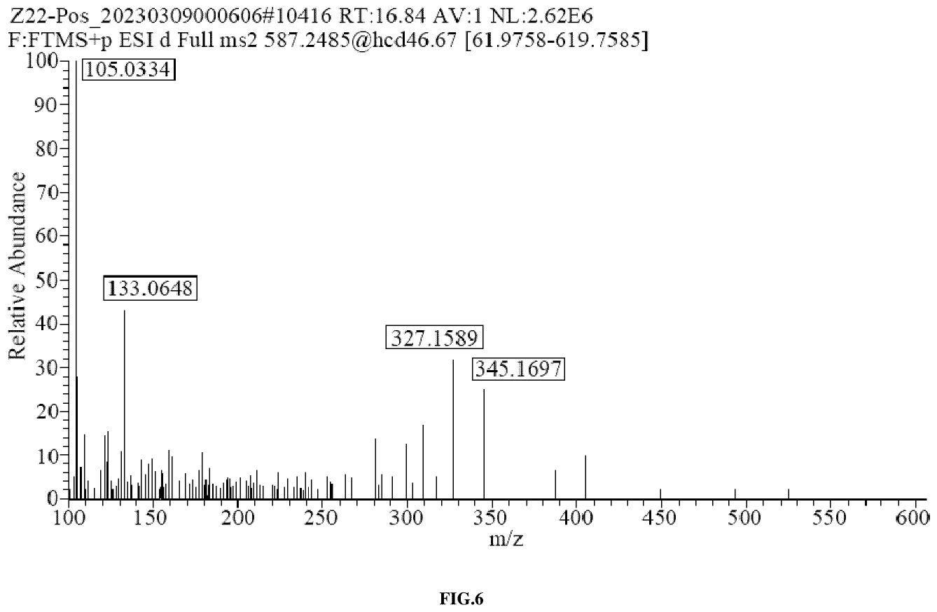

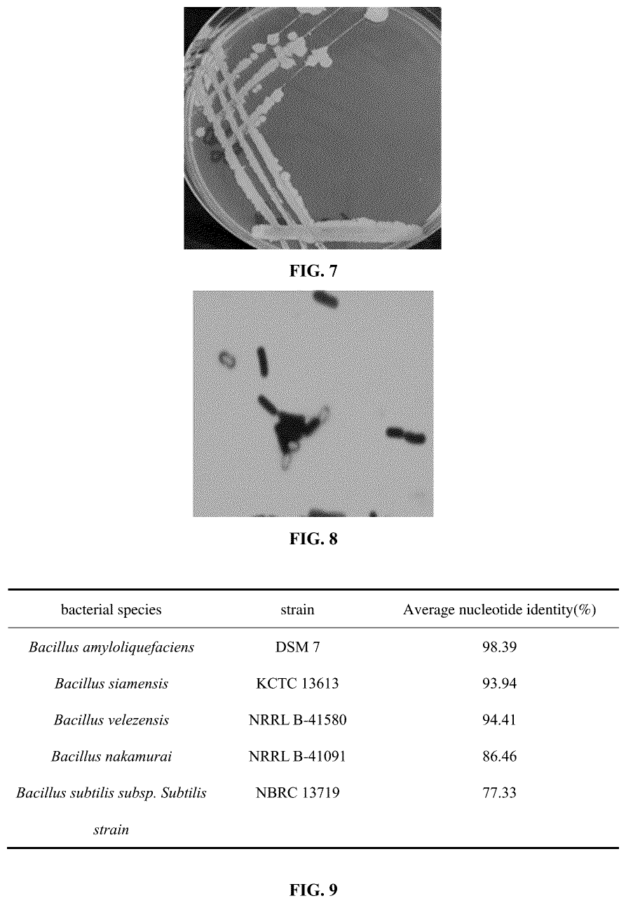

Morphological identification: the strain M-SHDS-22 was inoculated on an activation medium using a streak plate method and cultured at 32° C. to 35° C. for 32 h to 38 h until the plate was full of colonies. The colonies of the strain M-SHDS-22 after plate culture were shown in FIG. 7, where the colonies of the strain M-SHDS-22 were white, smooth and moist, and easy to be picked up. The Gram staining results of the colonies were shown in FIG. 8, indicating that the strain was a Gram-positive bacterium with a microscopic morphology of rod-shaped, and with central spores, no swelling.

Physiological and biochemical identification: the strain M-SHDS-22 showed positive results in both gelatinase test and VP test, and was able to ferment amygdalin.

### Molecular Biology Identification:

Sequencing analysis was conducted by Wuhan Tianyi Huayu Gene Technology Co., Ltd. The strain M-SHDS-22 had a 16S rRNA sequencing result set forth in SEQ ID NO: 1, a 16S rRNA gene sequencing BLAST result shown in Table 1.

SEQ ID NO: 1:
CAGATGGGAGCTTGCTCCCTGATGTTAGCGGCGGACGGGTGAGTAACAC
GTGGGTAACCTGCCTGTAAGACTGGGATAACTCCGGGAAACCGGGGCTA
ATACCGGATGCTTGTTTGAACCGCATGGTTCAGACATAAAAGGTGGCTT
CGGCTACCACTTACAGATGGACCCGCGGCGCATTAGCTAGTTGGTGAGG
TAACGGCTCACCAAGGCGACGATGCGTAGCCGACCTGAGAGGGTGATCG
GCCACACTGGGACTGAGACACGGCCCAGACTCCTACGGGAGGCAGCAGT
AGGGAATCTTCCGCAATGGACGAAAGTCTGACGGAGCAACGCCGCGTGA
GTGATGAAGGTTTTCGGATCGTAAAGCTCTGTTGTTAGGGAAGAACAAG
TGCCGTTCAAATAGGGCGGCACCTTGACGGTACCTAACCAGAAAGCCAC
GGCTAACTACGTGCCAGCAGCCGCGGTAATACGTAGGTGGCAAGCGTTG
TCCGGAATTATTGGGCGTAAAGGGCTCGCAGGCGGTTTCTTAAGTCTGA
TGTGAAAGCCCCCGGCTCAACCGGGGAGGGTCATTGGAAACTGGGGAAC
TTGAGTGCAGAAGAGGAGAGTGGAATTCCACGTGTAGCGGTGAAATGCG
TAGAGATGTGGAGGAACACCAGTGGCGAAGGCGACTCTCTGGTCTGTAA
CTGACGCTGAGGAGCGAAAGCGTGGGGAGCGAACAGGATTAGATACCCT
GGTAGTCCACGCCGTAAACGATGAGTGCTAAGTGTTAGGGGGTTTCCGC
CCCTTAGTGCTGCAGCTAACGCATTAAGCACTCCGCCTGGGGAGTACGG
TCGCAAGACTGAAACTCAAAGGAATTGACGGGGGCCCGCACAAGCGGTG
GAGCATGTGGTTTAATTCGAAGCAACGCGAAGAACCTTACCAGGTCTTG
ACATCCTCTGACAATCCTAGAGATAGGACGTCCCCTTCGGGGGCAGAGT
GACAGGTGGTGCATGGTTGTCGTCAGCTCGTGTCGTGAGATGTTGGGTT
AAGTCCCGCAACGAGCGCAACCCTTGATCTTAGTTGCCAGCATTCAGTT
GGGCACTCTAAGGTGACTGCCGGTGACAAACCGGAGGAAGGTGGGGATG
ACGTCAAATCATCATGCCCCTTATGACCTGGGCTACACACGTGCTACAA
TGGGCAGAACAAAGGGCAGCGAAACCGCGAGGTTAAGCCAATCCCACAA
ATCTGTTCTCAGTTCGGATCGCAGTCTGCAACTCGACTGCGTGAAGCTG
GAATCGCTAGTAATCGCGGATCAGCATGCCGCGGTGAATACGTTCCCGG
GCCTTGTACACACCGCCCGTCACACCACGAGAGTTTGTAACACCCGAAG
TCGGTGAGGTAACCTTTTTGGAGCCAGCCGCCGAAGGTGGGACAGATGA
TTGGG.

TABLE 1
BLAST result of 16S rRNA gene sequencing of strain M-SHDS-22

| | | | | Pairwise | Mismatch/ |
|---|---|---|---|---|---|
| Rank | Name | Strain | Accession | Similarity(%) | Total nt |
| 1 | Bacillus amyloliquefaciens | DSM 7 | FN597644 | 99.85975 | 2/1426 |
| 2 | Bacillus siamensis | KCTC 13613 | AJVF01000043 | 99.85975 | 2/1426 |
| 3 | Bacillus velezensis | CR-502 | AY603658 | 99.85369 | 2/1367 |
| 4 | Bacillus subtilis | NCIB 3610 | ABQL01000001 | 99.57924 | 6/1426 |
| 5 | Bacillus nakamurai | NRRL B-41091 | LSAZ01000028 | 99.57924 | 6/1426 |
| 6 | Bacillus cabrialesii | TE3 | MK462260 | 99.50912 | 7/1426 |
| 7 | Bacillus inaquosorum | KCTC 13429 | AMXN01000021 | 99.50912 | 7/1426 |
| 8 | Bacillus stercoris | JCM 30051 | MN536904 | 99.50912 | 7/1426 |
| 9 | Bacillus vallismortis | DV1-F-3 | JH600273 | 99.43899 | 8/1426 |
| 10 | Bacillus atrophaeus | JCM 9070 | AB021181 | 99.36886 | 9/1426 |
| 11 | Bacillus tequilensis | KCTC 13622 | AYTO01000043 | 99.36886 | 9/1426 |
| 12 | Bacillus halotolerans | ATCC 25096 | LPVF01000003 | 99.36886 | 9/1426 |
| 13 | Bacillus spizizenii | NRRL B-23049 | CP002905 | 99.36886 | 9/1426 |
| 14 | Bacillus mojavensis | RO-H-1 | JH600280 | 99.29874 | 10/1426 |
| 15 | Bacillus glycinifermentans | GO-13 | LECW01000063 | 98.52632 | 21/1425 |
| 16 | Bacillus paralicheniformis | KJ-16 | KY694465 | 98.45614 | 22/1425 |
| 17 | Bacillus licheniformis | ATCC 14580 | AE017333 | 98.24561 | 25/1425 |
| 18 | Bacillus haynesii | NRRL B-41327 | MRBL01000076 | 98.17544 | 26/1425 |
| 19 | Bacillus sonorensis | NBRC 101234 | AYTN01000016 | 98.10526 | 27/1425 |

As shown in Table 1, the strain M-SHIDS-22 belonged to the genus Bacillus sp. The strain M-SHDS-22 was compared with the strain whole genome data of Bacillus amyloliquefaciens (DSM7) model. The results were shown in FIG. 9, where an Average nucleotide identity (ANI) value was: 98.39%, which was greater than an ANI same-species determination threshold of 96%. After identification, the M-SHDS-22 was Bacillus amyloliquefaciens, named Bacillus amyloliquefaciens strain MHDS-1, and was deposited in the CCTCC in Wuhan University, China on Jul. 10, 2023, with a deposit number of CCTCC NO: M20231262.

### Example 2

### (1) Activation of the Strain:

The Bacillus amyloliquefaciens strain MHDS-1 was inoculated on LB solid medium and cultured at 32° C. for 38 h to obtain an activated Bacillus amyloliquefaciens strain MHDS-1.

The activation medium was an LB solid medium, including 10 g/L of peptone, 5 g/L of yeast extract, 10 g/L of sodium chloride, 15 g/L of agar, and water as a balance. Each component was dissolved by heating and then sterilized by high-pressure steam at 121° C. for 30 min.

### (2) Preparation of Bacterial Suspension:

Distilled water was filled into 6 15 mL centrifuge tubes, of which a 1st centrifuge tube was filled with 10 mL of the distilled water, while 2nd to 6th centrifuge tubes were filled with 9 mL of the distilled water, and the tube lids were not fastened. 100 mL of distilled water was filled into a 250 mL wide-mouthed triangular flask, and a breathable sealing film was placed on a flask mouth. After high-pressure steam sterilization at 121° C. for 30 min, the flasks were all placed in a super clean bench to allow air cooling to obtain sterile water.

The activated strain was scraped with a sterile inoculation loop and placed in the sterile water, and a strain concentration was adjusted to 0.5×10⁸ CFU/mL to obtain a bacterial suspension of the Bacillus amyloliquefaciens strain MHDS-1.

### (3) Fermentation:

80 g of 80-mesh Taxus media fine powder and 10 g of wheat bran were mixed to obtain a medical powder.

6 g of flour was added into 70 mL of water, boiled, and air-cooled to a room temperature to obtain a paste.

The paste was poured into the medical powder and blended evenly, where 0.4 mL of the bacterial suspension was added into per 100 g of a mixture of Taxus media fine powder, wheat bran, flour, and water to prepare a soft material, which was fermented at 29° C. under 65% humidity for 84 h, and then oven-dried at 55° C. to obtain a traditional Chinese medicine decoction piece of Taxus media.

### Example 3

### (1) Activation of the Strain:

The Bacillus amyloliquefaciens strain MHDS-1 was inoculated on LB solid medium and cultured at 33° C. for 36 h to obtain an activated Bacillus amyloliquefaciens strain MHDS-1.

The activation medium was an LB solid medium, including 10 g/L of peptone, 5 g/L of yeast extract, 10 g/L of sodium chloride, 15 g/L of agar, and water as a balance. Each component was dissolved by heating and then sterilized by high-pressure steam at 121° C. for 30 min.

### (2) Preparation of Bacterial Suspension:

Distilled water was filled into 6 15 mL centrifuge tubes, of which a 1st centrifuge tube was filled with 10 mL of the distilled water, while 2nd to 6th centrifuge tubes were filled with 9 mL of the distilled water, and the tube lids were not fastened. 100 mL of distilled water was filled into a 250 mL wide-mouthed triangular flask, and a breathable sealing film was placed on a flask mouth. After high-pressure steam sterilization at 121° C. for 30 min, the flasks were all placed in a super clean bench to allow air cooling to obtain sterile water.

The activated strain was scraped with a sterile inoculation loop and placed in the sterile water, and a strain concentration was adjusted to 1.5×10⁸ CFU/mL to obtain a bacterial suspension of the Bacillus amyloliquefaciens strain MHDS-1.

### (3) Fermentation:

120 g of 80-mesh Taxus media fine powder and 8 g of wheat bran were mixed to obtain a medical powder. 4 g of flour was added into 82 mL of water, boiled, and air-cooled to a room temperature to obtain a paste. The paste was poured into the medical powder and blended evenly, where 0.6 mL of the bacterial suspension was added into per 100 g of a mixture of Taxus media fine powder, wheat bran, flour, and water to prepare a soft material, which was fermented at 33° C. under 75% humidity for 60 h, and then oven-dried at 60° C. to obtain a traditional Chinese medicine decoction piece of Taxus media, where weights and volumes were in g for solids and mL for liquids.

### Example 4

### (1) Activation of the Strain:

The Bacillus amyloliquefaciens strain MHDS-1 was inoculated on LB solid medium and cultured at 35° C. for 32 h to obtain an activated Bacillus amyloliquefaciens strain MHDS-1.

The activation medium was an LB solid medium, including 10 g/L of peptone, 5 g/L of yeast extract, 10 g/L of sodium chloride, 15 g/L of agar, and water as a balance. Each component was dissolved by heating and then sterilized by high-pressure steam at 121° C. for 30 min.

### (2) Preparation of Bacterial Suspension:

Distilled water was filled into 6 15 mL centrifuge tubes, of which a 1st centrifuge tube was filled with 10 mL of the distilled water, while 2nd to 6th centrifuge tubes were filled with 9 mL of the distilled water, and the tube lids were not fastened. 100 mL of distilled water was filled into a 250 mL wide-mouthed triangular flask, and a breathable sealing film was placed on a flask mouth. After high-pressure steam sterilization at 121° C. for 30 min, the flasks were all placed in a super clean bench to allow air cooling to obtain sterile water.

The activated strain was scraped with a sterile inoculation loop and placed in the sterile water, and a strain concentration was adjusted to 1×10⁸ CFU/mL to obtain a bacterial suspension of the Bacillus amyloliquefaciens strain MHDS-1.

### (3) Fermentation:

100 g of 80-mesh Taxus media fine powder and 20 g of wheat bran were mixed to obtain a medical powder. 10 g of flour was added into 110 mL of water, boiled, and air-cooled to a room temperature to obtain a paste. The paste was poured into the medical powder and blended evenly, where 0.5 mL of the bacterial suspension was added into per 100 g of a mixture of Taxus media fine powder, wheat bran, flour, and water to prepare a soft material, which was fermented at 31° C. under 70% humidity for 72 h, and then oven-dried at 65° C. to obtain a traditional Chinese medicine decoction piece of Taxus media, where weights and volumes were in g for solids and mL for liquids.

### Comparative Example 1

80 g of 80-mesh Taxus media fine powder and 10 g of wheat bran were mixed to obtain a medical powder.

6 g of flour was added into 70 mL of water, boiled, and air-cooled to a room temperature to obtain a paste.

The paste was poured into the medical powder and blended evenly, a resulting product was fermented at 29° C. under 65% humidity for 84 h, and then oven-dried at 55° C. to obtain a traditional Chinese medicine decoction piece of Taxus media.

### Comparative Example 2

120 g of 80-mesh Taxus media fine powder and 8 g of wheat bran were mixed to obtain a medical powder. 4 g of flour was added into 82 mL of water, boiled, and air-cooled to a room temperature to obtain a paste. The paste was poured into the medical powder and blended evenly, a resulting product was fermented at 33° C. under 75% humidity for 60 h, and then oven-dried at 60° C. to obtain a traditional Chinese medicine decoction piece of Taxus media.

### Comparative Example 3

100 g of 80-mesh Taxus media fine powder and 20 g of wheat bran were mixed to obtain a medical powder. 10 g of flour was added into 110 mL of water, boiled, and air-cooled to a room temperature to obtain a paste. The paste was poured into the medical powder and blended evenly, a resulting fermentation medium was fermented at 31° C. under 70% humidity for 72 h, and then oven-dried at 65° C. to obtain a traditional Chinese medicine decoction piece of Taxus media.

### Test Example 1

The Taxus media raw products before fermentation and processing and the fermented Taxus media products prepared in Examples 2 to 4 and Comparative Examples 1 to 3 were used as test samples. An octadecylsilane-bonded silica gel column was used as a chromatographic column, with a column length of 250 mm, an inner diameter of 4.6 mm, and a particle size of 5 μm. The samples to be tested were subjected to gradient elution, with mobile phase A being 0.1% phosphoric acid water and mobile phase B being acetonitrile, at column temperature set at 30° C., a flow rate set at 1 mL/min, and an injection volume set at 10 μL. The procedure of the gradient elution was as follows:

0 min to 15 min: mobile phase A was 65% to 45%, mobile phase B was 35% to 55%;

15 min to 22.5 min: mobile phase A was 45% to 30%, mobile phase B was 55% to 70%;

22.5 min to 32.5 min: mobile phase A was 30% to 10%, mobile phase B was 70% to 90%; and

32.5 min to 35 min: mobile phase A was 10% to 65%, mobile phase B was 90% to 35%.

An eluate passing the column was collected and detected at a wavelength of 232 nm to obtain a paclitaxel peak area Y.

An appropriate amount of paclitaxel was accurately weighed and dissolved in methanol to obtain a mother liquor of paclitaxel reference substance with a mass concentration of 163.30 μg/mL. 1 mL, 0.75 mL, 0.5 mL, 0.25 mL, 0.05 mL, and 0.01 mL of the mother liquor of paclitaxel reference substance were separately accurately taken and diluted to 1 mL, and an injection volume was fixed at 10 μL. A standard curve was drawn with the reference substance concentration as a horizontal axis (X) and the peak area as a vertical axis (Y). A regression equation of the paclitaxel standard curve was finally obtained as Y=14,965.98X+0.58, with a linear range of 1.63-163.30 μg/mL. The peak area Y of paclitaxel obtained from the liquid phase result was substituted into the equation to calculate the X value to obtain the paclitaxel content, and the results were shown in Table 2.

TABLE 2
Determination results of paclitaxel content

| | Paclitaxel | Paclitaxel | |
|---|---|---|---|
| | content in | content in | |
| | raw product | fermentation | |
| | (μg/g) | product (μg/g) | Increase/% |
| Example 2 | 130.34 | 159.75 | 22.57 |
| Example 3 | 132.82 | 168.05 | 26.53 |
| Example 4 | 120.97 | 146.84 | 21.39 |
| Comparative | 138.94 | 150.23 | 8.12 |
| Example 1 | | | |
| Comparative | 130.67 | 142.81 | 9.29 |
| Example 2 | | | |
| Comparative | 141.55 | 150.21 | 6.12 |
| Example 3 | | | |

Table 2 showed that the Bacillus amyloliquefaciens strain MHDS-1 could effectively increase the paclitaxel content in Taxus media after processing, which could be increased by 21.39% to 26.53% compared with that of the Taxus media raw product. Compared with traditional natural fermentation, the processing method of Taxus media was more conducive to increasing the paclitaxel content in Taxus media.

It can be seen from the above examples that the Bacillus amyloliquefaciens strain MHDS-1 and the preparation method of Taxus media-based traditional Chinese medicine decoction piece of the present disclosure can effectively increase the content of anti-tumor active ingredients in Taxus media and alleviate the shortage of Taxus media resources.

Although the above examples have described the present disclosure in detail, they are only a part of, not all of, the examples of the present disclosure. Other examples may also be obtained by persons based on the examples without creative efforts, and all of these examples shall fall within the protection scope of the present disclosure.

1-10. (canceled)

11. A Bacillus amyloliquefaciens strain MHDS-1, wherein the Bacillus amyloliquefaciens strain MHDS-1 is deposited in the China Center for Type Culture Collection (CCTCC) with a deposit number of CCTCC NO: M20231262.

12. A method for increasing a paclitaxel content of Taxus media, comprising subjecting the Taxus media to fermentation with the Bacillus amyloliquefaciens strain MHDS-1 according to claim 11.

13. A processing method of Taxus media, comprising subjecting the Taxus media to fermentation with the Bacillus amyloliquefaciens strain MHDS-1 according to claim 11.

14. The processing method according to claim 13, comprising:
mixing an activated Bacillus amyloliquefaciens strain MHDS-1, the Taxus media, wheat bran, flour, and water to allow the fermentation.

15. The processing method according to claim 14, wherein the activated Bacillus amyloliquefaciens strain MHDS-1 is prepared by inoculating the Bacillus amyloliquefaciens strain MHDS-1 on an activation medium, and culturing at 32° C. to 35° C. for 32 h to 38 h.

16. The processing method according to claim 14, wherein a total mass of the Taxus media, the wheat bran, the flour, and the water and a viable count of the Bacillus amyloliquefaciens strain MHDS-1 are at a ratio of 100 g:0.2×10⁸ CFU to −100 g:0.9×10⁸ CFU;
the Taxus media, the wheat bran, the flour, and the water are at a dosage ratio of 80-120 g:8-20 g:4-10 g:70-110 mL; and
the fermentation is conducted at 29° C. to 33° C. under a humidity of 65% to 75% for 60 h to 84 h.

17. A traditional Chinese medicine decoction piece of Taxus media prepared by drying a fermentation product processed by the processing method according to claim 13.

18. The traditional Chinese medicine decoction piece of the Taxus media according to claim 17, wherein the drying is conducted at 55° C. to 65° C.

19. A method for preparing an anti-tumor product, comprising using the traditional Chinese medicine decoction piece of the Taxus media according to claim 17.

20. The traditional Chinese medicine decoction piece of Taxus media prepared by drying a fermentation product processed by the processing method according to claim 17, comprising: mixing an activated Bacillus amyloliquefaciens strain MHDS-1, the Taxus media, wheat bran, flour, and water to allow the fermentation.

21. The traditional Chinese medicine decoction piece of Taxus media prepared by drying a fermentation product processed by the processing method according to claim 17, wherein the activated Bacillus amyloliquefaciens strain MHDS-1 is prepared by inoculating the Bacillus amyloliquefaciens strain MHDS-1 on an activation medium, and culturing at 32° C. to 35° C. for 32 h to 38 h.

22. The traditional Chinese medicine decoction piece of Taxus media prepared by drying a fermentation product processed by the processing method according to claim 17, wherein a total mass of the Taxus media, the wheat bran, the flour, and the water and a viable count of the Bacillus amyloliquefaciens strain MHDS-1 are at a ratio of 100 g:0.2×10⁸ CFU to −100 g:0.9×10⁸ CFU;
the Taxus media, the wheat bran, the flour, and the water are at a dosage ratio of 80-120 g:8-20 g:4-10 g:70-110 mL; and
the fermentation is conducted at 29° C. to 33° C. under a humidity of 65% to 75% for 60 h to 84 h.

23. The method for preparing an anti-tumor product according to claim 19, wherein, the drying is conducted at 55° C. to 65° C.


SEQUENCE LISTING (1 sequence)

SEQ ID NO: 1 - DNA, 1426 bp, synthetic construct
  source 1..1426: mol_type = other DNA; note = 16S rRNA sequence of strain M-SHDS-22
cagatgggagcttgctccctgatgttagcggcggacgggtgagtaacacgtgggtaacctgcctgtaagactgggataactccgggaaaccggggctaataccggatgcttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttttggagccagccgccgaaggtgggacagatgattggg